(19) United States
(12) Patent Application Publication
(10) Pub. No.: US 2011/0053262 A1
(43) Pub. Date: Mar. 3, 2011

(54) METHODS OF FABRICATING ENHANCED TISSUE-ENGINEERED CARTILAGE

(76) Inventors: Kyriacos A. Athanasiou, Houston, TX (US); Benjamin Daniel Elder, Houston, TX (US); Jerry Hu, Houston, TX (US); Roman M. Natoli, Houston, TX (US); Christopher Morton Revell, Houston, TX (US)

(21) Appl. No.: 12/874,803

(22) Filed: Sep. 2, 2010

Related U.S. Application Data
(63) Continuation of application No. PCT/US2009/035712, filed on Mar. 2, 2009.
(60) Provisional application No. 61/033,094, filed on Mar. 3, 2008.

(51) Int. Cl. C12N 5/00; C12N 5/02

(52) U.S. Cl. 435/325; 435/383

(57) ABSTRACT

Compositions and methods for fabricating a tissue-engineered cartilage construct comprising: providing a cell sample comprising a plurality of chondrocytes; culturing the cell sample to produce a tissue-engineered cartilage construct; and treating the tissue-engineered cartilage construct, wherein treating the tissue-engineered cartilage construct comprises the use of a biochemical reagent, a mechanical force, hydrostatic pressure, or any combination thereof.

### CROSS-REFERENCE TO RELATED APPLICATIONS

This application is a continuation of International Application No. PCT/US2009/035712, filed Mar. 2, 2009, which claims the benefit of U.S. Provisional Patent Application Ser. No. 61/033,094, filed Mar. 3, 2008, the entire disclosures of which are incorporated by reference.

### STATEMENT OF GOVERNMENT INTEREST

The present invention was made with support under Grant Number R01 AR053286 awarded by the National Institutes of Health. The U.S. government has certain rights in the invention.

### BACKGROUND

The inability of cartilage to repair itself leads to a myriad of clinical conditions that are burdensome to both patient and society. Tissue engineering (TE) is one promising approach to reduce this burden through in vitro growth of neotissue followed by implantation.

One challenge of TE is to create tissue that has biomechanical properties similar to those of healthy native tissue so that the implanted construct can function under native conditions (environment, mechanical load, etc.). Such biomechanical properties include, among other things, the macroscopic functional representation of the tissue's underlying structure and biochemical content.

Efforts in articular cartilage TE thus far have created constructs with glycosaminoglycan (GAG) content and resulting compressive stiffness near functional levels. However, native collagen content and resulting tensile properties remain a challenge.

### SUMMARY

The present disclosure, in certain embodiments, relates generally to methods of fabricating tissue engineered constructs. In particular, the present disclosure, in certain embodiments, relates to improved methods of fabricating tissue-engineered cartilage.

In certain embodiments, the present disclosure provides a method of fabricating a tissue-engineered cartilage construct comprising providing a cell sample comprising a plurality of chondrocytes, culturing the cell sample to produce a tissue-engineered cartilage construct, and treating the tissue-engineered cartilage construct, wherein treating the tissue-engineered cartilage construct comprises the use of a biochemical reagent, a mechanical force, hydrostatic pressure, or any combination thereof.

In certain embodiments, the present disclosure provides a method of treating a tissue-engineered cartilage construct comprising providing a tissue-engineered cartilage construct and treating the tissue-engineered cartilage construct, wherein treating the tissue-engineered cartilage construct comprises the use of a biochemical reagent, a mechanical force, hydrostatic pressure, or any combination thereof.

### DRAWINGS

Some specific example embodiments of the disclosure may be understood by referring, in part, to the following description and the accompanying drawings.

FIG. 1 shows an example of a self-assembly process for fabricating tissue-engineered cartilage constructs.

FIG. 2 shows representative gross and histological pictures of self-assembled tissue constructs for all groups in C-ABC treatment example: 2 wk Control (A-E), 2 wk C-ABC treated (F-J), 4 wk Control (K-O), 4 wk C-ABC treated (P-T). Ruler markings=1 mm in A, F, K, P, and the scale bar=200 μm in E applies to all histological images. Note the return of GAG staining in C-ABC treated constructs at 4 wks (Q) and the absence of type I collagen staining in all treatment groups (C,H,M,R).

FIG. 3 shows plots of Total and type II collagen from the C-ABC treatment example. Total collagen was significantly increased following C-ABC treatment at 4 wks (* significantly different from control, p<0.05). Additionally, collagen type II has also been shown to significantly increase.

FIG. 4 shows a plot of construct stiffness and permeability from the C-ABC treatment example. The aggregate modulus (HA) of C-ABC treated constructs recovered to be equivalent to untreated constructs at 4 wks. Permeability (k) at 4 wks was significantly decreased with C-ABC treatment (*,† significantly different from control at respective time point, p<0.05).

FIG. 5 shows a plot of tensile modulus and ultimate tensile strength from the C-ABC treatment example. Both the apparent Young's modulus (EY) and ultimate tensile strength (UTS) were significantly increased (47 and 78% at 4 wks, respectively) following C-ABC treatment at both time points studied (*,† significantly different from control at respective time point, p<0.05).

FIG. 6 shows that HP treatment significantly increases aggregate modulus (HA) and Young's modulus (EY). Parallel increases in GAG/WW and collagen/WW were found. No differences were found in construct gross morphology or cellularity. Collagen II production was seen, with no collagen I production when immunohistochemistry was performed. No differences were found between the two control groups (i.e., between those bagged but not subject to pressure and those kept in Petri dishes).

FIG. 7 shows that HP application was found to be a significant factor in affecting HA, EY, GAG/WW, and collagen/WW. HP application from 10-14 days had greatest effect on construct properties. With HP application, 2.4-fold higher HA, 1.4-fold higher GAG/WW, 1.6-fold higher EY, and 1.4-fold higher collagen/WW were found.

FIG. 8 shows a plot of construct wet weight at 2 weeks (FIG. 8a) and 4 weeks (FIG. 8b), which was found to increase with application of direct compression.

FIG. 9 shows a plot of construct thickness at 2 weeks (FIG. 9a) and 4 weeks (FIG. 9b), which was found to increase with application of direct compression.

FIG. 10 shows a plot of construct stiffness, as indicated by the aggregate modulus (HA), which was found to increase significantly with application of direct compression.

FIG. 11 shows that the combination of treatment with HP and TGF-131 resulted in a synergistic positive effect on collagen per WW and Young's modulus over controls.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, in certain embodiments, relates generally to methods of fabricating tissue engineered constructs. In particular, the present disclosure, in certain embodiments, relates to improved methods of fabricating tissue-engineered cartilage.

In certain embodiments, the present disclosure provides a method of fabricating a tissue-engineered cartilage construct comprising providing a cell sample comprising a plurality of chondrocytes, culturing the cell sample to produce a tissue-engineered cartilage construct, and treating the tissue-engineered cartilage construct, wherein treating the tissue-engineered cartilage construct comprises the use of a biochemical reagent, a mechanical force, hydrostatic pressure, or any combination thereof.

In certain embodiments, the present disclosure provides a method of treating a tissue-engineered cartilage construct comprising providing a tissue-engineered cartilage construct and treating the tissue-engineered cartilage construct, wherein treating the tissue-engineered cartilage construct comprises the use of a biochemical reagent, a mechanical force, hydrostatic pressure, or any combination thereof.

The cells and cell samples used in conjunction with the methods of the present disclosure may comprise chondrocytes, chondro-differentiated cells, fibrochondrocytes, fibrochondro-differentiated cells, and combinations thereof (referred to herein as chondrocytes).

The chondrocytes may comprise articular chondrocytes. Generally, the articular chondrocytes may be from a bovine or porcine source, or another animal source. Alternatively if the construct is to be used for in vivo tissue replacement, the source of articular chondrocytes may be autologous cartilage from a small biopsy of the patient's own tissue, provided that the patient has healthy articular cartilage that may be used as the start of in vitro expansion. Another suitable source of chondrocytes is allogenic chondrocytes, such as those from histocompatible cartilage tissue obtained from a donor or cell line. The fibrochondrocytes used in conjunction with the methods of the present disclosure may comprise meniscal fibrochondrocytes. Generally, the meniscal fibrochondrocytes may be from a bovine or porcine source, or another suitable animal source, for in vitro studies. Alternatively if the construct is to be used for in vivo tissue replacement, the source of meniscal fibrochondrocytes may be autologous fibrocartilage from a small biopsy of the patient's own tissue, provided that the patient has healthy meniscal fibrocartilage that may be used as the start of in vitro expansion. Another suitable source of fibrochondrocytes is allogenic fibrochondrocytes, such as for example from histocompatible fibrocartilaginous tissue obtained from a donor or cell line.

In certain embodiments, the chondrocytes used in conjunction with the methods of the present disclosure may be derived from mesenchymal, embryonic, induced pluripotent stem cells, skin cells, or other stem cells.

The cells and cell samples may be derived from any source and site for obtaining a cell sample comprising a sufficient number of chondrocytes to produce a tissue-engineered cartilage construct. One of ordinary skill in the art, with the benefit of this disclosure, will recognize additional sources and sites from which to obtain a cell sample which may be suitable for use in the methods of the present invention.

Such cells and cell samples may be obtained by any means suitable for obtaining a cell sample comprising a sufficient number of chondrocytes to produce a tissue-engineered cartilage construct. In certain embodiments, such a means may comprise enzymatic digestion of native tissue. Suitable enzymes for such an enzymatic digestion include, but are not limited to, one or more collagenases.

The cells and cell samples may be cultured using any suitable means and conditions to produce a tissue-engineered cartilage construct. Choices in such means and conditions include, but are not limited to, the seeding concentration of the cell sample, the medium in which the cell sample is cultured, and the shape of the vessel in which the cell sample is cultured. The choice of such conditions may depend upon, among other things, the source of the cell sample and the desired size and shape of the tissue-engineered cartilage construct. One of ordinary skill in the art, with the benefit of this disclosure, will recognize suitable means and conditions for producing tissue-engineered cartilage constructs useful in the methods of the present invention.

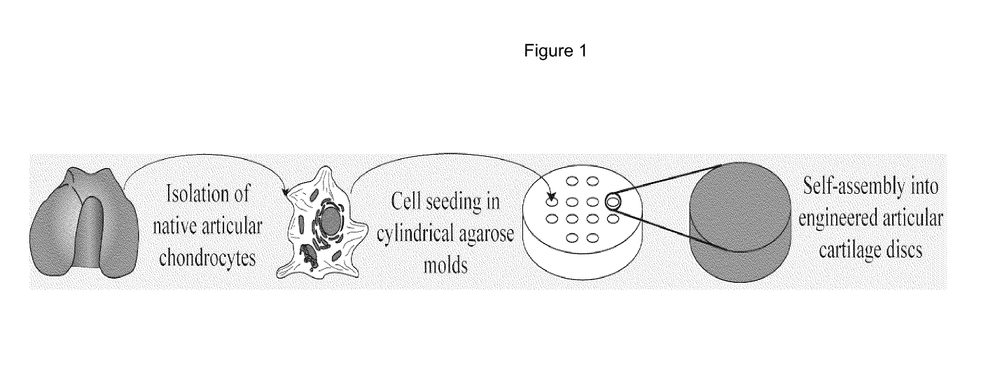

In certain embodiments, the culturing of the cell sample to produce a tissue-engineered cartilage construct may utilize a self-assembly process. An example of such a self-assembly process is shown in FIG. 1. In this exemplary self-assembly process, the cell sample is cultured under suitable conditions in a cylindrical agarose mold to produce disc-shaped tissue-engineered cartilage constructs.

The step of treating the tissue-engineered cartilage construct may be performed at any desired time, which may be during or after the tissue-engineered cartilage construct is produced. In certain embodiments, treating the tissue-engineered cartilage construct may comprise the use of a biochemical reagent, a mechanical force, hydrostatic pressure, or any combination thereof. Such treatments may, among other things, enhance the morphological, biochemical, and/or biomechanical properties of the treated tissue-engineered cartilage construct.

A variety of biochemical reagents may be used to treat the tissue-engineered cartilage constructs. Such biochemical reagents include any biochemical reagent suitable for enhancing the morphological, biochemical, and/or biomechanical properties of the treated tissue-engineered cartilage construct. Such suitable biochemical reagents may include, but are not limited to, gylcosaminoglycan (GAG) depleting agents, growth factors, and any combination thereof. Example of GAG depleting agents which may be suitable for use in the methods of the present invention are chondroitinase-ABC (C-ABC), aggrecanases, keratinases, NaCl or Guanidinium-HCl extraction, and combinations thereof. An example of a growth factor which may be suitable for use in the methods of the present invention is transforming growth factor-β1 (TGF-β1). One of ordinary skill in the art, with the benefit of this disclosure, may recognize additional biochemical reagents that may be useful in the methods of the present invention. The biochemical reagents useful in the methods of the present invention may be used to treat the tissue-engineered cartilage constructs at any time during or after the production of the tissue-engineered cartilage construct. Such a choice of treatment time may depend upon, among other things, the desired degree of treatment and the specific biochemical reagent chosen. One of ordinary skill in the art, with the benefit of this disclosure, will be able to choose when to treat the tissue-engineered cartilage construct with the biochemical reagents useful in the methods of the present invention.

In certain embodiments, a treatment using a GAG depleting agent may comprise treating the tissue-engineered cartilage construct with practically protease-free C-ABC at an activity of 2 U/mL media for 4 hours at 37° C. By way of explanation, and not of limitation, such a treatment may, among other things, substantially remove GAGs from the tissue-engineered cartilage construct, and following a period of culture after this one-time GAG depleting agent treatment, total collagen concentration may increase, GAGs may be produced, and tensile properties, such as the apparent Young's modulus, may increase. In certain embodiments, such improvements may occur without a substantial increase in compressive stiffness of the tissue-engineered cartilage construct.

In certain embodiments, the GAG depleting agent concentration used to treat the tissue-engineered cartilage construct may vary from 0.001 U/mL to 5 U/mL. In certain embodiments, the time of the GAG depleting agent treatment may be varied between 0.01 hrs up to 4 weeks. In certain embodiments, the GAG depleting agent treatment may be applied at varying time points during and/or after the production of the tissue-engineered cartilage construct. In certain embodiments, the GAG depleting agent may be applied repeatedly as opposed to a one-time treatment. Such variations, among other things, may result in varying degrees of GAG depletion and may aid in the enhancement of the morphological, biochemical, and/or biomechanical properties of the treated tissue-engineered cartilage construct. For example, treatment with C-ABC at 2 weeks and 4 weeks has affected decorin and resulted in 3.4 MPa of Young's modulus at 6 wks.

The mechanical force used in the methods of the present invention to treat the tissue-engineered cartilage construct may be applied in any amount and by any means suitable to enhance the morphological, biochemical, and/or biomechanical properties of the treated tissue-engineered cartilage construct. An example of a suitable mechanical force is direct compression. In certain embodiments, the choice of an appropriate mechanical force may comprise the selection of an appropriate strain and frequency. Such a choice of strain and frequency may depend upon, among other things, the size and shape of the tissue-engineered cartilage construct. One of ordinary skill in the art, with the benefit of this disclosure, will recognize suitable strains and frequencies that may be useful in the methods of the present invention.

In certain embodiments, the use of mechanical force may comprise the use of a strain of 7 to about 17% and a frequency of 0 to about 1 Hz. In certain embodiments, such mechanical force may be applied from 1 to 4 days after production of the tissue-engineered cartilage construct in 60 second cycles (i.e. 60 seconds of mechanical force, followed by 60 seconds of no mechanical force) for about 1 hour total mechanical force application per day. By way of explanation, and not of limitation, such a mechanical force treatment may, among other things, increase one or more of the wet weight (ww), thickness, and ratio of GAG concentration to wet weight (GAG/ww) of the tissue-engineered cartilage construct.

In certain embodiments, the mechanical force treatment may be applied with a varying (i.e. non-repetitive) manner, such as varying periods in which no mechanical force is applied. In certain embodiments, the mechanical force may be applied on non-consecutive days. In certain embodiments, the mechanical force may be applied at differing strains ranging from about 0.1% to about 99%. In certain embodiments, mechanical forces of various magnitudes may be applied during the same treatment. Such variations in the mechanical force treatment, among other things, may aid in the enhancement of the morphological, biochemical, and/or biomechanical properties of the treated tissue-engineered cartilage construct.

The hydrostatic pressure (HP) used in the methods of the present invention to treat the tissue-engineered cartilage construct may be applied in any amount and by any means suitable to enhance the morphological, biochemical, and/or biomechanical properties of the treated tissue-engineered cartilage construct. In certain embodiments, the HP used in the methods of the present invention may be static HP. In certain embodiments, the choice of an appropriate HP may comprise the choice of an appropriate magnitude and duration of HP treatment. One of ordinary skill in the art, with the benefit of this disclosure, will recognize suitable magnitudes and durations of HP treatment that may be useful in the methods of the present invention.

In certain embodiments, the use of hydrostatic pressure to treat the tissue-engineered cartilage construct may comprise the use of 10 MPa static HP for 1 hour/day for a 5-day period before or after the production of the tissue-engineered cartilage construct. In certain embodiments, such a hydrostatic pressure treatment may increase one or more of the aggregate modulus, the Young's modulus, the ratio of GAGs to wet weight (GAG/ww), and the ratio of collagen to wet weight (collagen/ww).

In certain embodiments, hydrostatic pressure may be applied repeatedly on non-consecutive days. In certain embodiments, hydrostatic pressure may be applied multiple times per day, optionally with varying periods in which no hydrostatic pressure is applied. In certain embodiments, the magnitude of the hydrostatic pressure may range from about 0.01 to about 20 MPa. In certain embodiments, varying magnitudes of hydrostatic pressure may be utilized in the same treatment. In certain embodiments, non-static HP may be employed, optionally at varying frequencies. In certain embodiments, such non-static HP treatments may have a sinusoidal pattern of magnitude.

In certain embodiments, the tissue-engineered cartilage constructs may be treated with a treatment comprising a combination of one or more of biochemical reagents, mechanical forces, and hydrostatic pressure. For example, the combined treatment of the tissue-engineered cartilage construct may comprise treatment with TGF-β1 and 10 MPa of static hydrostatic pressure (the latter for 1 hour per day for 5 days after production of the tissue-engineered cartilage construct). Such a combined treatment, among other things, may result in a synergistic positive effect on collagen/ww and Young's modulus.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### EXAMPLES

### C-ABC Treatment of Tissue-Engineered Cartilage Constructs

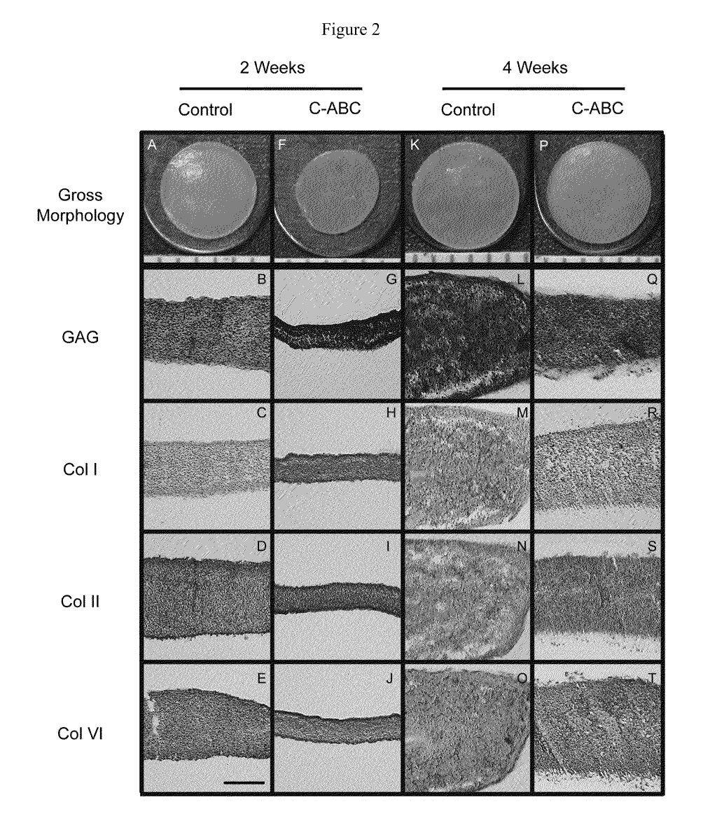

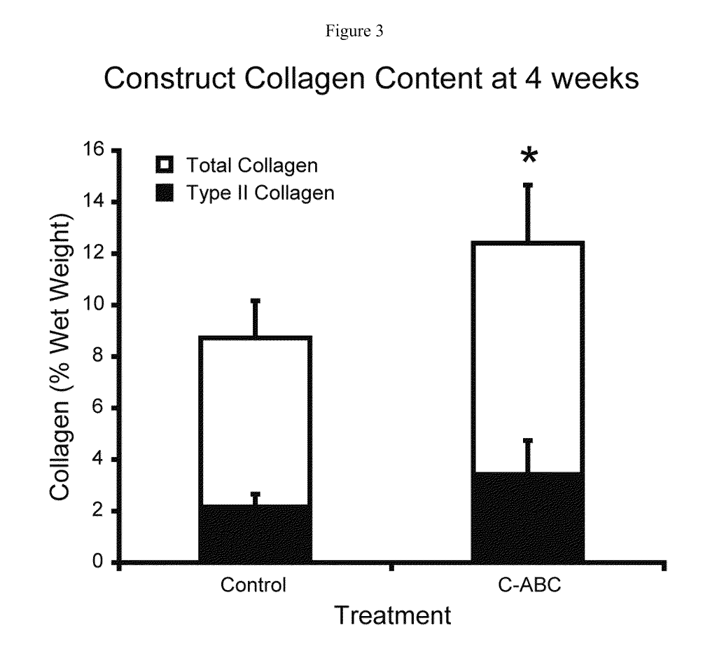

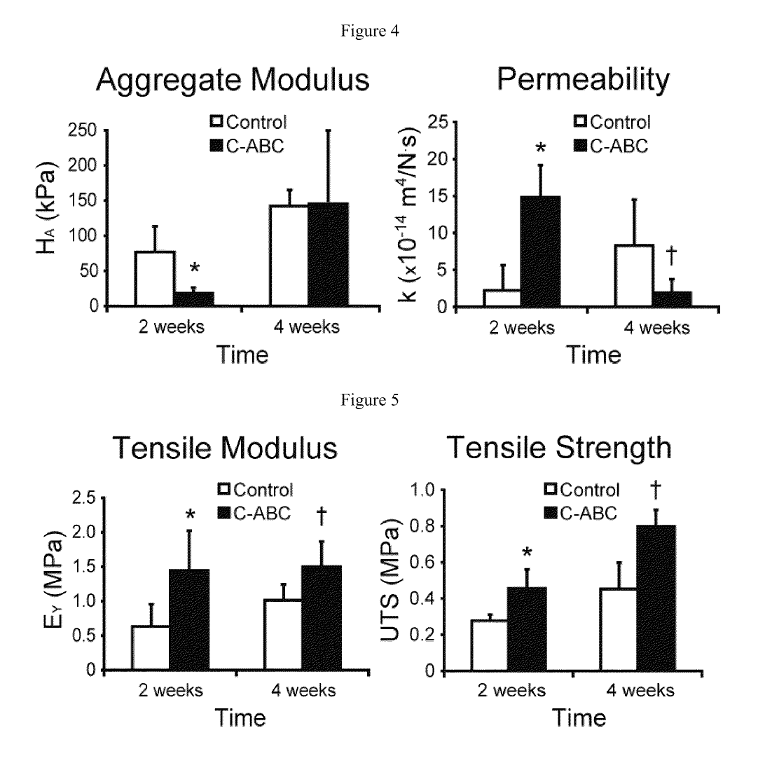

Tissue engineered constructs were treated with protease-free C-ABC (Sigma) at an activity of 2 U/mL media for 4 hrs at 37° C. Post-treatment constructs were thoroughly washed five times with 400 mL of fresh media. C-ABC treatment resulted in elimination of glycosaminoglycans (GAG) from the construct (FIG. 2). After 2 weeks of culture following this one-time C-ABC treatment, total collagen concentration was increased at 4 weeks in the C-ABC treated groups compared to no treatment, though total collagen content, and type II collagen content and concentration, were not significantly different (FIG. 3). Further, GAGs returned (FIG. 2), and the compressive stiffness of treated constructs and untreated controls was similar (FIG. 4). Tensile properties were increased by 47 and 78% for the apparent Young's modulus and ultimate tensile strength, respectively (FIG. 5).

Hydrostatic Pressure Treatment of Tissue-Engineered Cartilage Constructs

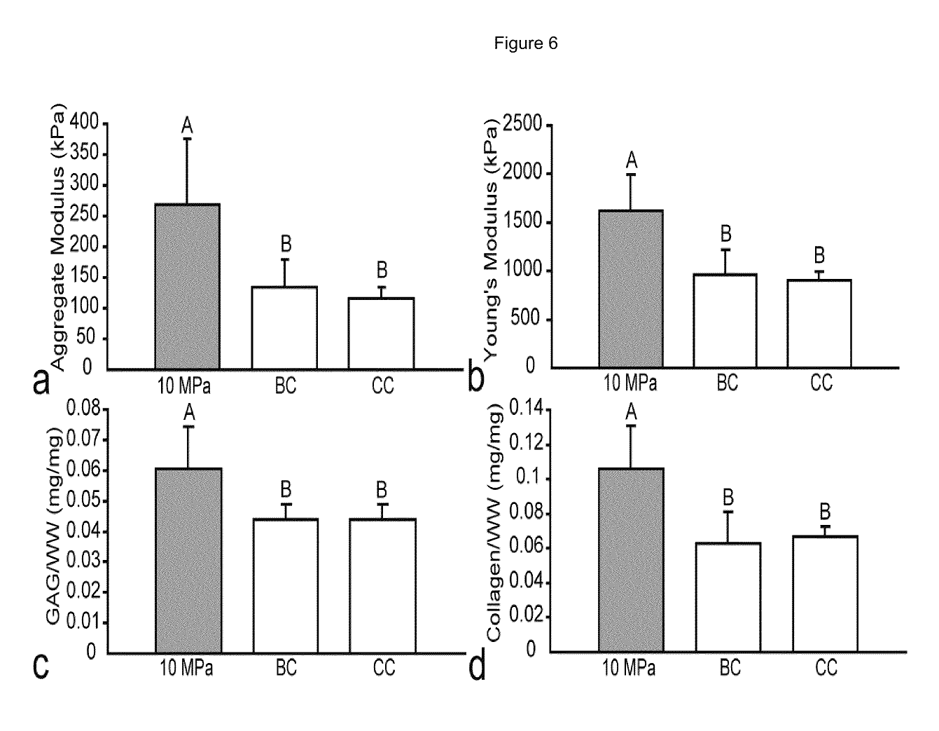

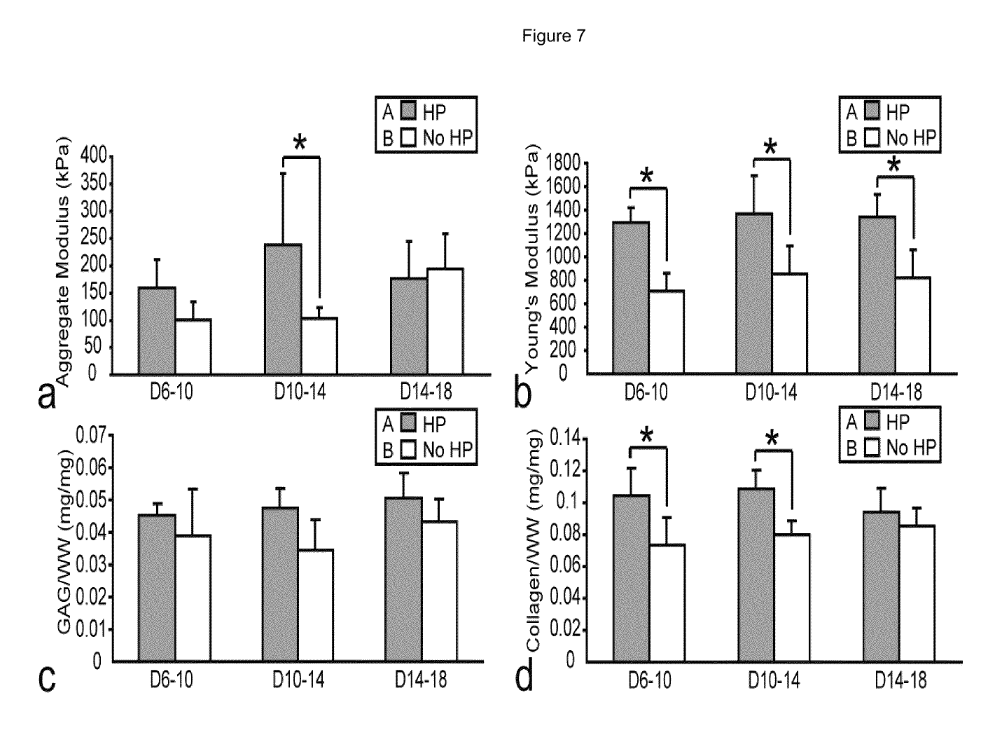

10 MPa static HP was applied for 1 hour/day to tissue engineered constructs on t=6-10, t=10-14, t=14-18 days from initial seeding. It was observed that HP application from 10-14 days had greatest effect on construct properties, resulting in 2.4-fold higher aggregate modulus (HA), 1.4-fold higher GAG/ww, 1.6-fold higher Young's modulus (EY), and 1.4-fold higher collagen/ww (FIGS. 6 and 7).

Direct Compression Treatment of Tissue-Engineered Cartilage Constructs

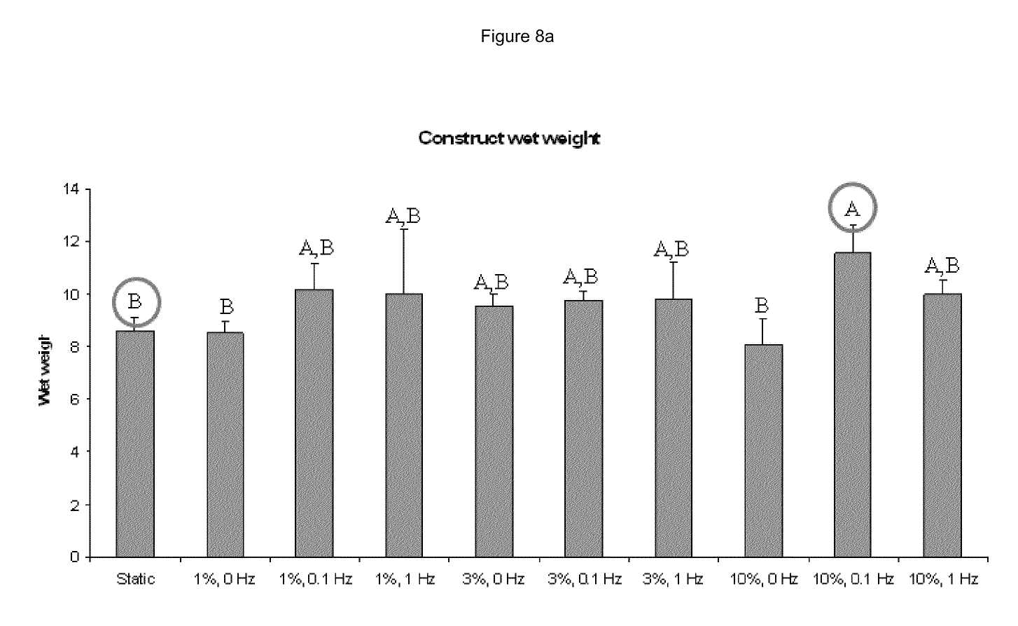

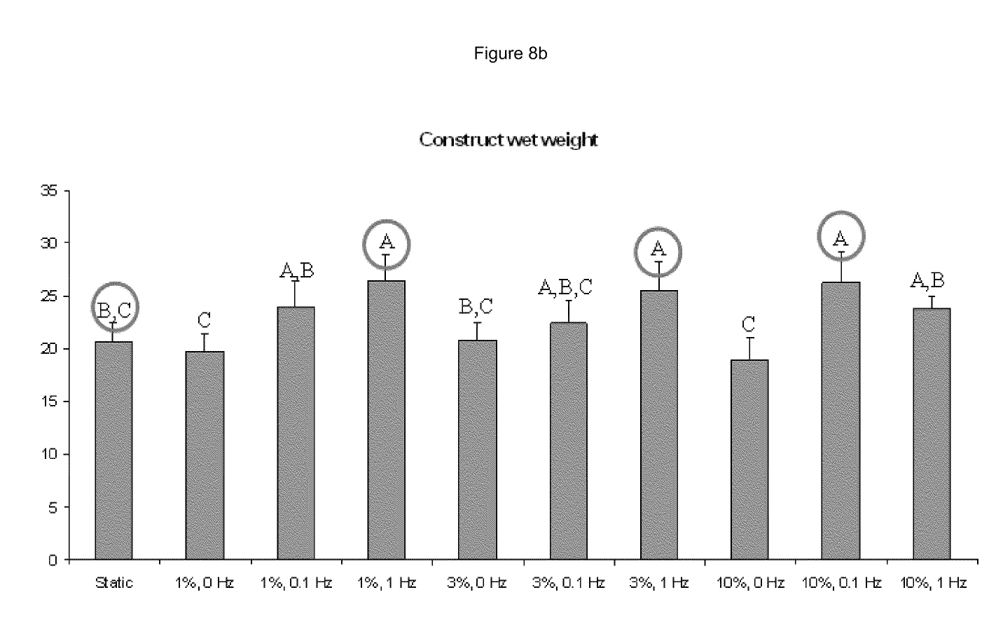

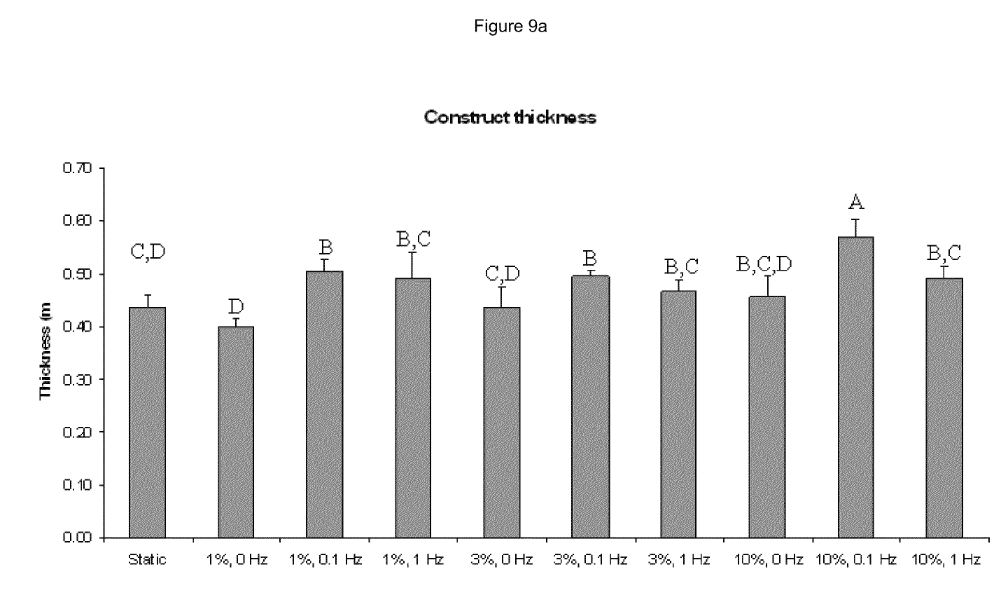

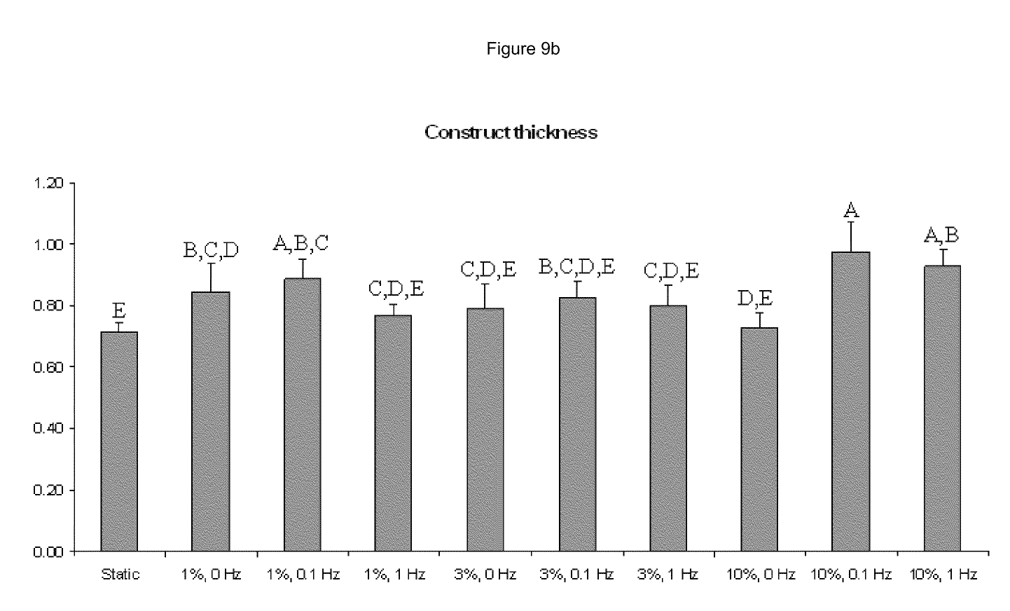

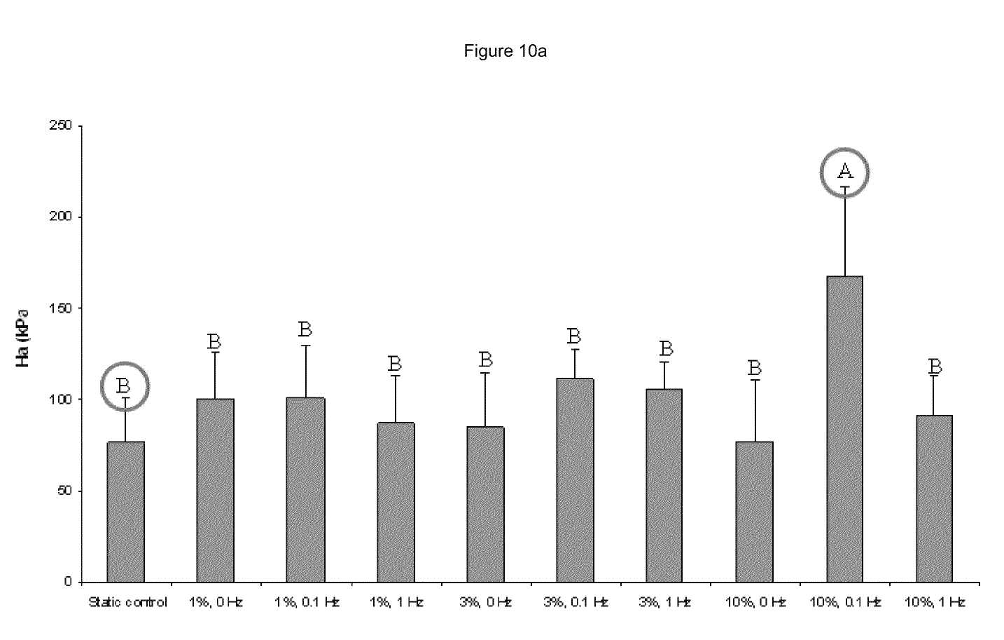

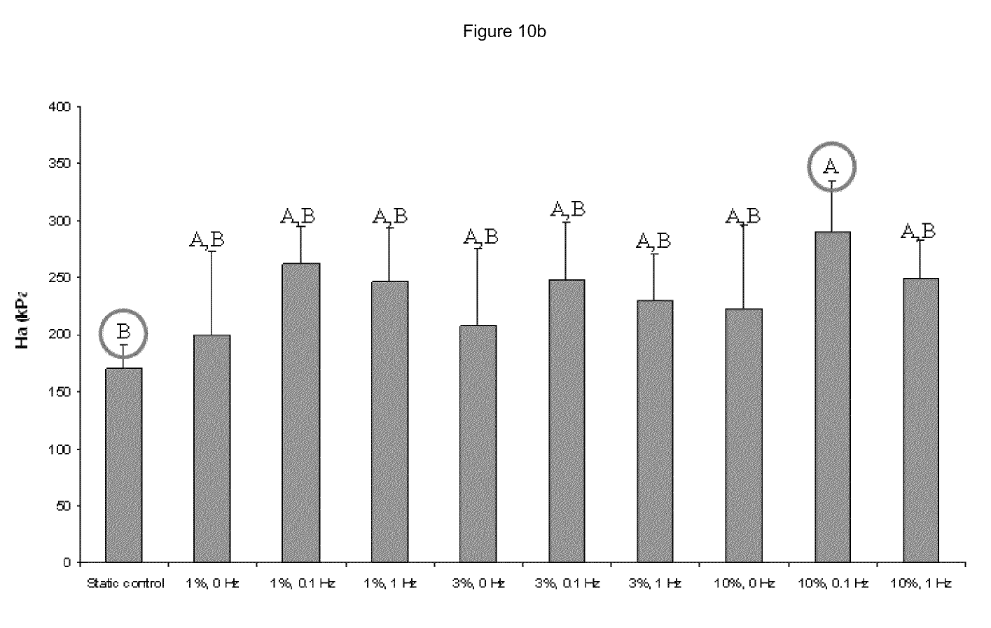

Direct compression (DC) at 7, 10, and 17% strain and 0, 0.1, and 1 Hz was applied from days 11-14 post-seeding, in 60 second cycles (i.e. 60 seconds of direct compression, followed by 60 seconds of no direct compression) for 1 hour total compression per day. Morphologically, DC application resulted in significant increases in wet weight (FIG. 8) and thickness (FIG. 9). Fifteen days post-seeding, the 17%, 0.1 Hz regimen yielded constructs with significant increase in HA, though all other treatments trended higher (FIG. 10). At this time, all regimens were also found to significantly increase GAG/ww except 17%, 1 Hz. The 17%, 0.1 Hz regimen was specifically found to significantly improve mechanical properties.

Combination Treatment of Tissue-Engineered Cartilage Constructs

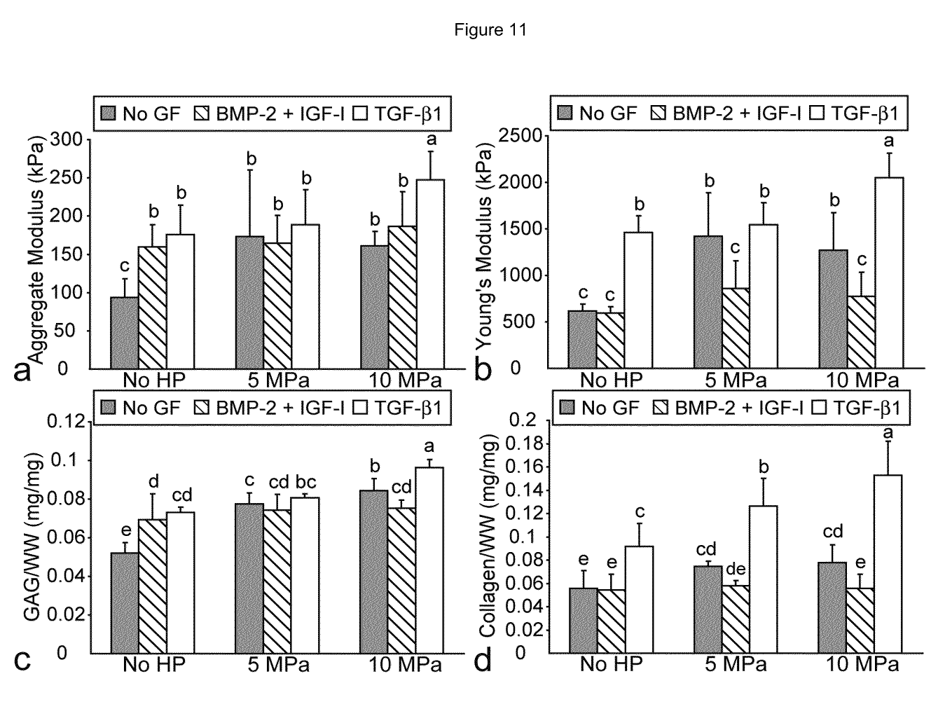

For the use of combined effects of growth factors and HP, the combined application of TGF-β1 and 10 MPa of static hydrostatic pressure (the latter for 1 hour per day from days 10-14 post-seeding) resulted in a synergistic positive effect on collagen/ww and Young's modulus over controls (FIG. 11).

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method for fabricating a tissue-engineered cartilage construct comprising:
providing a cell sample comprising a plurality of chondrocytes;
culturing the cell sample to produce a tissue-engineered cartilage construct; and
treating the tissue-engineered cartilage construct, wherein treating the tissue-engineered cartilage construct comprises the use of a biochemical reagent, a mechanical force, hydrostatic pressure, or any combination thereof.

2. The method of claim 1 wherein the biochemical reagent is selected from the group consisting of a glycosaminoglycan depleting agent, a growth factor, and any combination thereof.

3. The method of claim 1 wherein the biochemical reagent is selected from the group consisting of chondroitinase-ABC, TGF-β1, and any combination thereof.

4. The method of claim 1 wherein the mechanical force is direct compression.

5. The method of claim 1 wherein the hydrostatic pressure is static hydrostatic pressure.

6. The method of claim 1 wherein the hydrostatic pressure is non-static hydrostatic pressure.

7. The method of claim 6 wherein the non-static hydrostatic pressure has a sinusoidal pattern of magnitude.

8. A method for treating a tissue-engineered cartilage construct comprising:
providing a tissue-engineered cartilage construct; and
treating the tissue-engineered cartilage construct, wherein treating the tissue-engineered cartilage construct comprises the use of a biochemical reagent, a mechanical force, hydrostatic pressure, or any combination thereof.

9. The method of claim 8 wherein the biochemical reagent is selected from the group consisting of a glycosaminoglycan depleting agent, a growth factor, and any combination thereof.

10. The method of claim 8 wherein the biochemical reagent is selected from the group consisting of chondroitinase-ABC, TGF-β1, and any combination thereof.

11. The method of claim 8 wherein the mechanical force is direct compression.

12. The method of claim 8 wherein the hydrostatic pressure is static hydrostatic pressure.

13. The method of claim 12 wherein the hydrostatic pressure is non-static hydrostatic pressure.

14. The method of claim 13 wherein the non-static hydrostatic pressure has a sinusoidal pattern of magnitude.

15. A tissue-engineered cartilage construct formed by the method of claim 1.

16. A tissue-engineered cartilage construct formed by the method of claim 8.
